(19) United States
(12) Patent Application Publication
(10) Pub. No.: US 2024/0170157 A1
(43) Pub. Date: May 23, 2024

(54) RESUPPLY ATTRITION USING MACHINE LEARNING

(71) Applicant: Brightree LLC, Peachtree Corners, GA (US)

(72) Inventors: Fadi Kamal HADDAD, San Diego, CA (US); Kiran THOMAS, Cumming, GA (US)

(21) Appl. No.: 18/508,888

(22) Filed: Nov. 14, 2023

Related U.S. Application Data
(60) Provisional application No. 63/427,005, filed on Nov. 21, 2022.

(51) Int. Cl. G16H 50/30; G16H 10/60

(52) U.S. Cl. CPC G16H 50/30; G16H 10/60

(57) ABSTRACT

Techniques for patient medical care are disclosed. These techniques include identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient. The techniques further include predicting a probability of attrition from a medical treatment for the patient based on providing the prediction data to a machine learning (ML) model, where the ML model is trained to predict the probability using prior patient medical data and prior patient order data, relating to a plurality of prior patients. The techniques further include triggering an intervention to prophylactically discourage attrition from the medical treatment for the patient, based on the predicted probability of attrition.

### CROSS-REFERENCE TO RELATED APPLICATIONS

This application claims priority to U.S. Provisional Patent Application No. 63/427,005, filed Nov. 21, 2022, the entire content of which is incorporated herein by reference in its entirety.

### INTRODUCTION

Aspects of the present disclosure relate to patient medical care, and more specifically, to improved prediction of patients at risk for attrition from treatment.

Many medical patients undergo treatment for an extended duration. Treatment can be ongoing for weeks, months, or years. Patients undergoing extended treatment are at risk for prematurely discontinuing their treatment, commonly termed attrition. Identifying patients that are at risk for attrition is extremely important, because identifying these patients can allow a care provider to take actions to reduce the risk of attrition. Reducing the risk of attrition, and continuing treatment, can significantly improve patient outcomes. But identifying patients that are at risk for attrition is typically not viable using existing techniques.

### SUMMARY

Embodiments include a method. The method includes identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient. The method further includes predicting a probability of attrition from a medical treatment for the patient based on providing the prediction data to a machine learning (ML) model, where the ML model is trained to predict the probability using prior patient medical data and prior patient order data, relating to a plurality of prior patients. The method further includes triggering an intervention to prophylactically discourage attrition from the medical treatment for the patient, based on the predicted probability of attrition.

Embodiments further include an apparatus, including a memory and a hardware processor communicatively coupled to the memory, the hardware processor configured to perform operations. The operations include identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient. The operations further include predicting a probability of attrition from a medical treatment for the patient based on providing the prediction data to an ML model, where the ML model is trained to predict the probability using prior patient medical data and prior patient order data, relating to a plurality of prior patients. The operations further include triggering an intervention to prophylactically discourage attrition from the medical treatment for the patient, based on the predicted probability of attrition.

Embodiments further include a non-transitory computer-readable medium comprising instructions that, when executed by a processor, cause the processor to perform operations. The operations include identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient. The operations further include predicting a probability of attrition from a medical treatment for the patient based on providing the prediction data to an ML model, where the ML model is trained to predict the probability using prior patient medical data and prior patient order data, relating to a plurality of prior patients. The operations further include triggering an intervention to prophylactically discourage attrition from the medical treatment for the patient, based on the predicted probability of attrition.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for predicting patient attrition using machine learning (ML), according to one embodiment.

FIG. 2 depicts a block diagram for a controller for predicting patient attrition using ML, according to one embodiment.

FIG. 3 is a flowchart illustrating predicting patient attrition using ML, according to one embodiment.

FIG. 4 is a flowchart illustrating inferring a prediction of patient attrition using ML, according to one embodiment.

FIG. 5 is a flowchart illustrating training an ML model for prediction of patient attrition, according to one embodiment.

FIG. 6 illustrates acting on an attrition prediction, according to one embodiment.

FIG. 7 illustrates parsing data using natural language processing (NLP) for predicting patient attrition using ML, according to one embodiment.

FIG. 8 depicts patient characteristics for use in predicting patient attrition using an ML model, according to one embodiment.

FIG. 9 depicts order history characteristics for use in predicting patient attrition using an ML model, according to one embodiment.

FIG. 10 depicts intervention history characteristics for use in predicting patient attrition using an ML model, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved prediction of patient attrition. As discussed above, identifying patients at risk for attrition from a medical treatment is extremely challenging. Existing systems typically do not include viable techniques to identify patients at risk for attrition, prior to the patients discontinuing medical treatment. Instead, patients are identified after they have discontinued treatment.

This is inefficient and harmful to patient outcomes. It is very difficult to encourage patients to resume treatment, after they have discontinued treatment. Further, after patients have discontinued treatment they are not benefiting from the therapeutic outcomes from the treatment, which can significantly harm health outcomes.

In aspects described herein, a probability of patient attrition from a medical treatment can be identified automatically using a trained ML model, based on prediction data relating to the patient. For example, the prediction data can include patient medical data, patient order history data, and patient intervention data. A suitable ML model (e.g., a random forest ML model) can be trained using historical data (e.g., historical patient medical data, historical patient order data, and historical patient intervention data). The trained ML model can then predict a probability of attrition, for a given patient, based on prediction data for that patient.

In an embodiment, this probability of attrition can be used to prophylactically intervene and discourage attrition for the patient. For example, the probability can be compared to a threshold value. If the probability exceeds the threshold, an intervention can be triggered, including an automatic communication with the patient, a care provider for the patient, or a medical facility for the patient. This intervention can discourage attrition, helping to keep the patient continuing the medical treatment and improving health outcomes for the patient.

Thus, aspects described herein provide significant advantages. First, as noted above identifying patients at risk for attrition significantly improves patient treatment and improves health outcomes. By identifying patients at risk of discontinuing treatment, and encouraging those patients to remain in treatment, the patients are more likely to continue to receive the therapeutic effects of the treatment, improving patient health outcomes.

Second, predicting a probability of attrition automatically using a trained ML model provides for an accurate prediction while minimizing the needed computational resources for the prediction and shifting the computational burden from prediction time (e.g., when near real-time response may be needed) to an earlier training time (e.g., when resources can be easily dedicated to the training). In an embodiment, one could attempt to identify patients at risk for attrition using a specific rubric or algorithm with pre-defined rules. This is likely to be inaccurate and non-viable, in practice, but to the extent it could be done it would also be computationally expensive, because a very large number of rules would be needed and parsing and following the rules is computationally expensive. Further, this computationally expensive analysis would done at the time the patient is evaluated for potential attrition, when a rapid response is likely to be needed (e.g., so that intervention can happen quickly).

Predicting a probability of attrition automatically using a trained ML model, by contrast, is significantly less computationally expensive at the time the probability is generated. For example, the prediction ML model can be trained up-front during a training phase, when rapid response is not necessary and computational resources are readily available. The trained ML model can then be used to rapidly, and computationally relatively cheaply, predict attrition for the patient. This provides a significant technical advantage over prior techniques by shifting the computational burden from the prediction time, when a rapid response is needed and computational resources may be engaged in other tasks, to a planned training time when a rapid response is not necessary and computational resources are available.

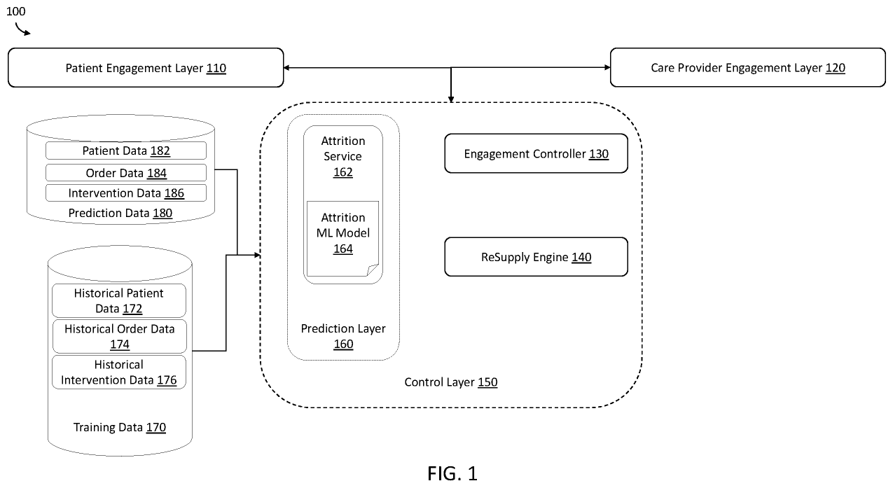

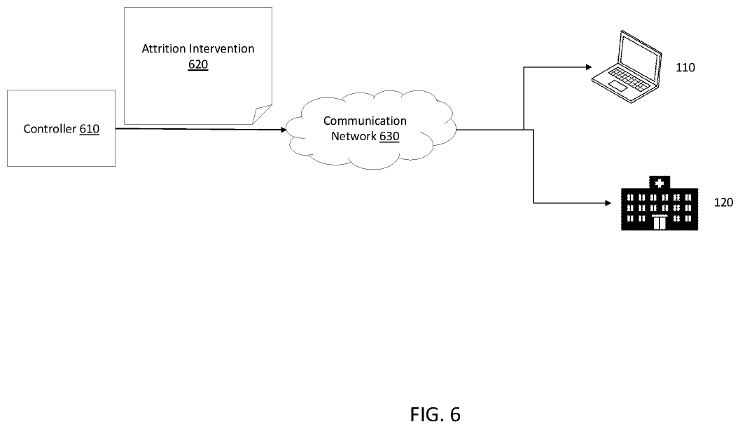

FIG. 1 depicts a computing environment 100 for predicting patient attrition using machine learning (ML), according to one embodiment. In an embodiment the computing environment 100 includes a patient engagement layer 110. The patient engagement layer 110 facilitates engagement with a patient (e.g., through electronic transmissions, telephone calls, and any other suitable techniques) for medical resupply, and for other purposes. For example, the patient engagement layer 110 can be used to communicate with a patient, or otherwise take an action for the patient, when the patient is predicted to be at risk for attrition from a medical treatment. This is discussed further, below, with regard to FIG. 6.

For example, the patient engagement layer 110 can facilitate transmitting electronic messages (e.g., short message service (SMS) messages, multimedia messaging service messages (MMS), e-mail messages, or any other suitable electronic messages) to a patient, and receiving electronic messages from a patient, to facilitate prophylactically reducing a predicted risk of attrition for the patient. This is merely one example.

In an embodiment, the patient engagement layer 110 communicates with a control layer 150 including an engagement controller 130 and a resupply engine 140. For example, the engagement controller 130 can control patient engagement to facilitate automated medical resupply. In an embodiment, the patient engagement layer 110 communicates with the engagement controller 130 using a suitable communication network, including the Internet, a wide area network, a local area network, or a cellular network, and uses any suitable wired or wireless communication technique (e.g., WiFi or cellular communication).

In an embodiment the computing environment 100 further includes a care provider engagement layer 120. The care provider engagement layer 120 facilitates engagement with a care provider (e.g., through electronic transmissions to an electronic healthcare system, telephone calls, and any other suitable techniques) for medical resupply, and other purposes. For example, the care provider engagement layer 120 can be used to communicate with a care provider regarding a patient, or otherwise to take an action for the patient through the care provider, when the patient is predicted to be at risk for attrition from a medical treatment. This is discussed further, below, with regard to FIG. 6.

In an embodiment, the care provider engagement layer 120 communicates with the engagement controller 130. For example, the engagement controller 130 can control care provider engagement to facilitate taking an action to prophylactically reduce a predicted risk of patient attrition. In an embodiment, the care provider engagement layer 120 communicates with the engagement controller 130 using a suitable communication network, including the Internet, a wide area network, a local area network, or a cellular network, and uses any suitable wired or wireless communication technique (e.g., WiFi or cellular communication).

In an embodiment, the engagement controller 130 interacts with the resupply engine 140. For example, the resupply engine 140 can interact with the engagement controller 130 to generate resupply events and facilitate resupply. This can include facilitating all suitable aspects of resupply, including ordering, shipping, billing, and any other suitable tasks.

Further, in an embodiment, the control layer 150 includes a prediction layer 160. The prediction layer 160 includes an attrition service 162 and an attrition ML model 164. In an embodiment, the attrition ML model 164 is a trained ML model. The attrition service 162 uses the attrition ML model 164 to predict a risk of attrition for a patient.

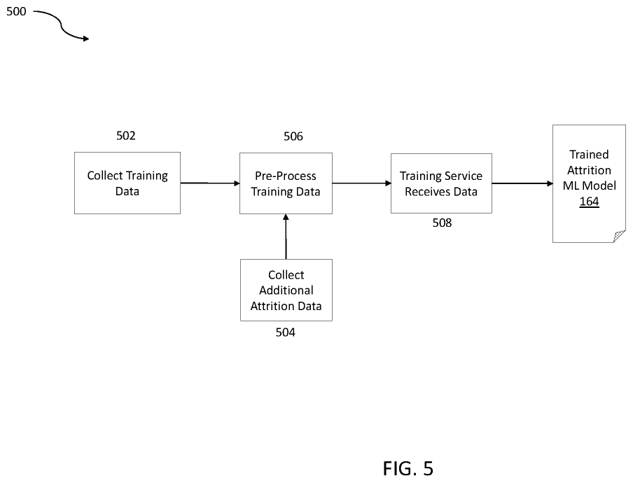

For example, the attrition ML model 164 can be trained using training data 170. This can include historical patient data 172, historical order data 174, and historical intervention data 176. As discussed further below with regard to FIG. 5, the training data 170 can be used to train the attrition ML model 164. For example, the attrition service 162 can provide the attrition ML model 164 with prediction data 180, and the attrition ML model 164 can use the prediction data 180 to infer an attrition risk score for a given patient. The prediction data 180 can include patient data 182, order data 184, and any other suitable data. This is discussed further, below, with regard to FIGS. 5 and 8-10. The attrition risk score can be used to predict a likelihood of attrition for a patient, and the engagement controller 130 can use the attrition risk score to attempt to mitigate the risk of attrition.

In an embodiment, the prediction data 180 and the training data 170 can be stored in one or more suitable electronic databases (e.g., a relational database, a graph database, or any other suitable database) or other electronic repositories (e.g., a cloud storage location, an on-premises network storage location, or any other suitable electronic repository). The prediction data 180 and the training data 170 can be provided from the respective electronic repositories to the prediction layer 160 using any suitable communication network, including the Internet, a wide area network, a local area network, or a cellular network, and can use any suitable wired or wireless communication technique (e.g., WiFi or cellular communication).

In an embodiment, the engagement controller 130 can identify how to contact a particular patient or care provider through queries to the patient engagement layer 110 and care provider engagement layer 120. For example, a patient engagement layer 110 can relate to multiple different healthcare entities with a large number of patients. The engagement controller 130 can determine how to contact a particular patient by querying each electronic healthcare system associated with the patient engagement layer (e.g., using a suitable identifier or identifiers associated with the patient), and receiving a response from the electronic healthcare system related to the patient (e.g., the patient's insurer, healthcare provider, or any other suitable healthcare entity). In an embodiment, the engagement controller 130 can determine whether the electronic healthcare system related to the patient engagement layer 110 is compatible with the control layer 150 (e.g., with the engagement controller 130, the resupply engine 140, or both). The engagement controller 130 can decline to interact further with an incompatible patient engagement layer 110. An electronic healthcare system that does not recognize the requested patient will not respond, or will respond with an indication that the patient is not recognized. An electronic healthcare system that recognizes the patient will respond with an indication that the patient is successfully recognized. The engagement controller 130 can use that successful response to determine how to engage with the patient in the future.

Similarly, the engagement controller 130 can determine how to contact a particular care provider by querying each electronic healthcare system associated with the care provider engagement layer (e.g., using a suitable identifier or identifiers associated with the care provider, including a national provider identifier (NPI)), and receiving a response from the electronic healthcare system related to the care provider (e.g., a healthcare entity where the care provider works). In an embodiment, the engagement controller 130 can determine whether the electronic healthcare system related to the care provider engagement layer 120 is compatible with the control layer 150 (e.g., with the engagement controller 130, the resupply engine 140, or both). The engagement controller 130 can decline to interact further with an incompatible care provider engagement layer 120. In an embodiment, an electronic healthcare system that does not recognize the requested care provider will not respond, or will respond with an indication that the care provider is not recognized. An electronic healthcare system that recognizes the care provider will respond with an indication that the care provider is successfully recognized. The engagement controller 130 can use that successful response to determine how to engage with the care provider in the future.

In an embodiment, the engagement controller 130 interacts with a single patient engagement layer 110 and a single care provider engagement layer 120 (e.g., each associated with multiple healthcare entities). Alternatively, or in addition, the engagement controller 130 can interact with multiple patient engagement layers 110 (e.g., each associated with a particular healthcare entity or small number of healthcare entities), multiple care provider engagement layers 120 (e.g., each associated with a particular healthcare entity or small number of healthcare entities), or both. In this example, the engagement controller 130 can query multiple patient engagement layers 110 and multiple care provider engagement layers 120, as appropriate, to determine how to contact the respective patient and care provider.

In an embodiment the patient engagement layer 110, the care provider engagement layer 120, the engagement controller 130, the resupply engine 140, and the prediction layer 160, can be implemented using any suitable combination of physical compute systems, cloud compute nodes and storage locations, or any other suitable implementation. For example, the patient engagement layer 110, the care provider engagement layer 120, the engagement controller 130, the resupply engine 140, and the prediction layer 160, could each be implemented using a respective server or cluster of servers. As another example, the patient engagement layer 110, the care provider engagement layer 120, the engagement controller 130, the resupply engine 140, and the prediction layer 160, can be implemented using a combination of compute nodes and storage locations in a suitable cloud environment. For example, one or more of the components of the patient engagement layer 110, the care provider engagement layer 120, the engagement controller 130, the resupply engine 140, and the prediction layer 160 can be implemented using a public cloud, a private cloud, a hybrid cloud, or any other suitable implementation.

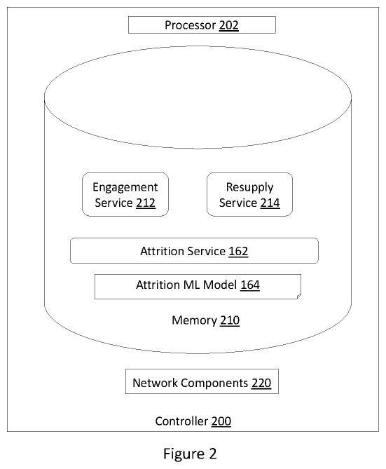

FIG. 2 depicts a block diagram for a controller 200 for predicting patient attrition using ML, according to one embodiment. The controller 200 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 200 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory associated with physical addresses, such as random access memory (RAM), read only memory (ROM), flash memory, or other types of volatile and/or non-volatile memory.

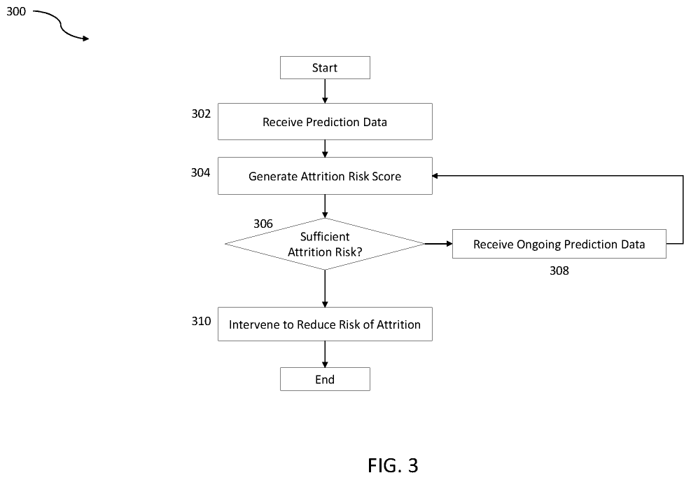

The memory 210 generally includes program code for performing various functions related to use of the engagement controller 130. The program code is generally described as various functional “applications” or “modules” within the memory 210, although alternate implementations may have different functions and/or combinations of functions. Within the memory 210, the engagement service 212 facilitates patient and care provider engagement (e.g., using the patient engagement layer 110 and care provider engagement layer 120 illustrated in FIG. 1). This is discussed further, below, with regard to FIG. 6. The resupply service 214 facilitates automated medical resupply. In an embodiment, the memory 210 further includes an attrition service 162 and an attrition ML model 164. In an embodiment, the attrition ML model 164 is a trained ML model. The attrition service 162 facilitates training the attrition ML model, and using the attrition ML model 164 to predict a risk of attrition for a patient. This is discussed further, below, with regard to FIGS. 3-5.

While the controller 200 is illustrated as a single entity, in an embodiment, the various components can be implemented using any suitable combination of physical compute systems, cloud compute nodes and storage locations, or any other suitable implementation. For example, the controller 200 could be implemented using a server or cluster of servers. As another example, the controller 200 can be implemented using a combination of compute nodes and storage locations in a suitable cloud environment. For example, one or more of the components of the controller 200 can be implemented using a public cloud, a private cloud, a hybrid cloud, or any other suitable implementation.

Although FIG. 2 depicts the engagement service 212, the resupply service 214, the attrition service 162, and the attrition ML model 164 as being separately located in the memory 210 that representation is also merely provided as an illustration for clarity. More generally, the controller 200 may include one or more computing platforms, such as computer servers for example, which may be co-located, or may form an interactively linked but distributed system, such as a cloud-based system, for instance. As a result, the processor 202, and the memory 210 may correspond to distributed processor and memory resources within the computing environment 100. Thus, it is to be understood that any, or all, of the engagement service 212, the resupply service 214, the attrition service 162, and the attrition ML model 164, may be stored together, or remotely from one another, within the distributed memory resources of the computing environment 100.

FIG. 3 is a flowchart 300 illustrating predicting patient attrition using ML, according to one embodiment. At block 302, an attrition service (e.g., the attrition service 162 illustrated in FIGS. 1-2) receives prediction data. For example, the attrition service can use an attrition ML model (e.g., the attrition ML model 164 illustrated in FIGS. 1-2) to predict a risk of attrition for a patient.

In an embodiment, the attrition ML model is a trained ML model. For example, the attrition ML model can be a random forest ML model. As another example, the attrition ML model can be a logistic progression ML model, a gradient boosting ML model (e.g., a light gradient boosting machine (light GBM) classifier), or a fully randomized trees ML model. These are merely examples, and the attrition ML model can be any suitable supervised ML model (e.g., a trained deep neural network (DNN), regression model, or any other suitable supervised ML model). Training the attrition ML model is discussed further below with regard to FIG. 5.

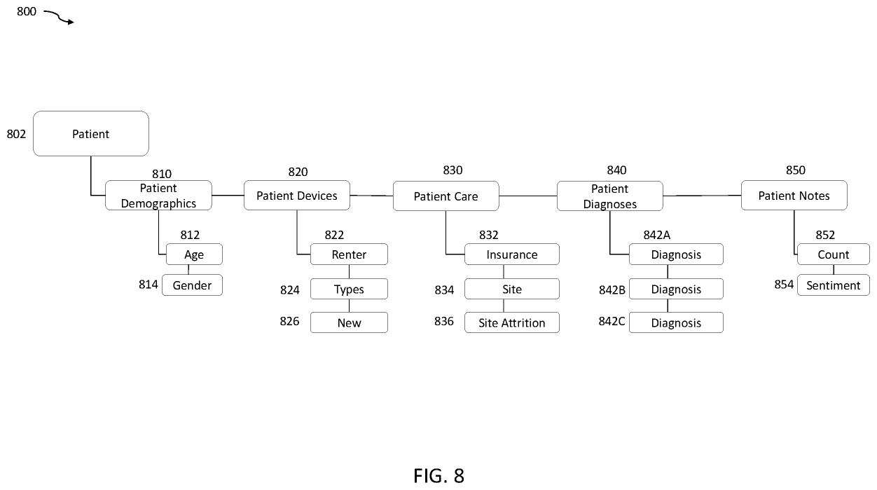

In an embodiment, the training data can include historical patient data (e.g., the historical patient data 172 illustrated in FIG. 1). This can include historical data about medical diagnoses and treatments for patients (e.g., the patients for whom historical order data is used), as well as patient demographic data, patient note data (e.g., unstructured textual data analyzed using sentiment analysis) and any other suitable data. The training data can further include historical order data (e.g., the historical order data 174 illustrated in FIG. 1). This can include historical data about resupply orders, devices, and payments for patients. The training data can further include The training data can further include historical intervention data (e.g., the historical intervention data 176 illustrated in FIG. 1). This can include phone interventions, e-mail interventions, text interventions, and any other suitable intervention data for patients. The training data is discussed further, below, with regard to FIGS. 8-10.

In an embodiment, the training data has had any personally identifying information (e.g., patient identifying information and any other personally identifying information) removed before being used to train the attrition ML model. Further, in an embodiment, the training data is data collected at different snapshots of time. For example, the training data can be collected using a lookback period (e.g., one month prior to the snapshot, 6 months prior to the snapshot, one year prior to the snapshot, or any other suitable period).

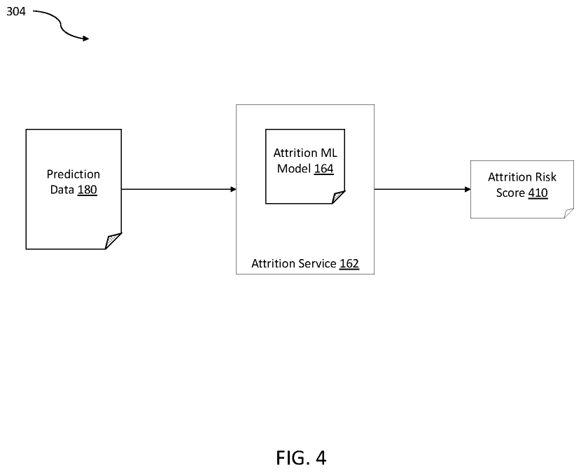

At block 304, the attrition service generates an attrition risk score. In an embodiment, the attrition service can use the trained attrition ML model to predict the attrition risk score (e.g., during inference). This is discussed further below with regard to FIG. 4. For example, the attrition service can provide the attrition ML model with prediction data (e.g., the prediction data 180 illustrated in FIG. 1), and the attrition ML model can use the prediction data to infer a probability of attrition for a given patient. In an embodiment, the prediction data includes patient data (e.g., the patient data 182 illustrated in FIG. 1), order data (e.g., the order data 184), and intervention data (e.g., the intervention data 186 illustrated in FIG. 1). The prediction data is discussed further, below, with regard to FIGS. 8-10.

In an embodiment, the attrition risk score indicates a probability that the subject patient will prematurely discontinue their treatment. In an embodiment, the attrition ML model can be trained to calculate this risk for a given time horizon (e.g., a week, a month, 90 days, 6 months, one year, or any other suitable time horizon). Alternatively, the attrition ML model can be trained to calculate this risk for the duration of the patient's treatment. In an embodiment, the attrition score is a number, or tuple of numbers, indicating the patient's risk of attrition. This is merely an example, and the attrition risk score can be any suitable value or collection of values (e.g., one or more Boolean values, textual values, or any other suitable values).

At block 306, the attrition service determines whether the attrition risk score indicates a sufficient risk of attrition to intervene take action to mitigate the risk. For example, the attrition service can determine whether the attrition risk score meets a threshold of attrition risk. This threshold can be generated manually (e.g., provided by a data scientist or administrator) or automatically. For example, an ML model (e.g., the attrition ML model or another suitable ML model) can be used to identify the threshold. As another example, the attrition risk score can itself indicate whether the attrition risk is sufficient for intervention. If the attrition risk score is not sufficient for intervention, the flow proceeds to block 308.

At block 308 the attrition service receives ongoing prediction data. For example, the attrition service can receive ongoing order data (e.g., indicating additional resupply orders, communications, or behaviors) and ongoing patient data (e.g., indicating changes to diagnoses or treatments for the patient). In an embodiment, the attrition service uses the ongoing prediction data to refine, and update, the prediction of attrition risk. For example, the attrition service can provide the ongoing prediction data to the attrition ML model to further predict an attrition risk score.

Returning to block 306, if the attrition risk score indicates a sufficient risk of attrition for intervene, the flow proceeds to block 310. At block 310, the attrition service intervenes to reduce the risk of attrition. This is discussed further, below, with regard to FIG. 6. In an embodiment, the attrition service engages with the patient to reduce the risk of attrition. This can include transmitting electronic messages to the patient (e.g., an SMS message, an e-mail message, a mobile App notification, or any other suitable electronic message) encouraging the patient to continue treatment. It can further include providing the patient with educational or training content (e.g., video information, audio information, or textual information) to assist the patient with treatment. The attrition service can further initiate telephone calls to the patient, or any other suitable engagement with the patient.

Alternatively, or in addition, the attrition service engages with a care provider for the patient. For example, the attrition service can provide an electronic message (e.g., an SMS message, an e-mail message, a mobile App notification, or any other suitable electronic message), initiate a telephone call, or take any other suitable action to notify the care provider about the risk of attrition and facilitate intervention by the care provider. The attrition service can further provide the care provider with data describing the patient's history (e.g., their resupply or medical history) to assist the care provider in intervening, and can provide the care provider with suggested actions to intervene and reduce the risk of attrition. These are merely examples, and the attrition service can intervene in any suitable manner to reduce the risk of attrition.

### Example of Predicting Attrition Risk Scores

FIG. 4 is a flowchart illustrating inferring a prediction of patient attrition using ML, according to one embodiment. In an embodiment, FIG. 4 provides one example of predicting attrition risk scores from prediction data (e.g., order data, patient data, and intervention data) using an ML model, discussed above in relation to block 304 illustrated in FIG. 3. Prediction data 180 (e.g., as discussed above in relation to FIG. 1) are provided to an attrition service 162 and an attrition ML model 164. In an embodiment, the prediction data 180 includes patient data (e.g., the patient data 182 illustrated in FIG. 1), order data (e.g., the order data 184), and intervention data (e.g., the intervention data 186 illustrated in FIG. 1).

In an embodiment, the attrition service 162 can use the attrition ML model 164 to predict an attrition risk score (e.g., during inference). As discussed above in relation to block 304 illustrated in FIG. 3, in an embodiment the attrition risk score 410 indicates a probability that the subject patient will prematurely discontinue their treatment. In an embodiment, the attrition ML model 164 can be trained to calculate this risk for a given time horizon (e.g., a week, a month, 90 days, six months, one year, or any other suitable time horizon). Alternatively, the attrition ML model can be trained to calculate this risk for the duration of the patient's treatment. In an embodiment, the attrition score is a number, or tuple of numbers, indicating the patient's risk of attrition. This is merely an example, and the attrition risk score can be any suitable value or collection of values (e.g., one or more Boolean values, textual values, or any other suitable values).

### Example of Training an Attrition ML Model

FIG. 5 is a flowchart 500 illustrating training an ML model for prediction of patient attrition, according to one embodiment. At block 502, a training service (e.g., a human administrator or a software or hardware service) collects training data. For example, an attrition service (e.g., the attrition service 162 illustrated in FIGS. 1 and 2) can be configured to act as the training service and collect training data (e.g., the training data 170 illustrated in FIG. 1). The training data can include historical patient data (e.g., the historical patient data 172 illustrated in FIG. 1), historical order data (e.g., the historical order data 174 illustrated in FIG. 1), and historical intervention data (e.g., the historical intervention data 176 illustrated in FIG. 1). Further, as discussed above in relation to block 302 illustrated in FIG. 3, the training data can be collected at different snapshots of time. For example, the training data can be collected using a lookback period (e.g., one month, six months, one year, or any other suitable duration prior to the snapshot).

At block 506, the training service (or other suitable service) pre-processes the collected training data. For example, the training service can create feature vectors reflecting the values of various features, for the training data. As another example the training service cleans and prepares the data for training the model. Some examples of cleaning the data include: identifying data that is not formatted properly and removing such data, identifying data with missing aspects and removing the data or updating the missing aspects with default values, identifying features that contain single or very few values and removing such values, identifying and removing duplicate data, and identifying and removing features that have very low correlation to the result. As another example, the training service uses time series processing to extract and process features from the data. At block 508, the training service receives the feature vectors and uses them to train an attrition ML model 164.

In an embodiment, at block 504 the training service also collects additional attrition data (e.g., data generated from patient or caregiver surveys or other human evaluations of treatment and attrition). At block 506, the training service can also pre-process this additional attrition data. For example, the feature vectors corresponding to the training data can be further annotated using the additional attrition data. Alternatively, or in addition, additional feature vectors corresponding to the additional retention data can be created. At block 508, the training service uses the pre-processed additional attrition data during training to generate the trained attrition ML model 164.

In an embodiment, while a variety of suitable data is available for the training service (e.g., the training data discussed with regard to block 502 and the additional attrition data discussed with regard to block 504), a subset of this data is selected to use for training of the attrition ML model 164. That is, as part of model design the training data is selected from an available universe of training data. Further, the attrition ML model 164 can then use the same, or similar, data types or fields for inference for inference (e.g., as discussed above with regard to FIG. 4). That is, as discussed further below in relation to FIGS. 8-10, in an embodiment the same data features are used for training and prediction.

In an embodiment, the pre-processing and training can be done as batch training. In this embodiment, all data is pre-processed at once (e.g., all historical retention data and additional retention data), and provided to the training service at block 508. Alternatively, the pre-processing and training can be done in a streaming manner. In this embodiment, the data is streaming, and is continuously pre-processed and provided to the training service. For example, it can be desirable to take a streaming approach for scalability. The set of training data may be very large, so it may be desirable to pre-process the data, and provide it to the training service, in a streaming manner (e.g., to avoid computation and storage limitations). Further, in an embodiment, a federated learning approach could be used in which multiple healthcare entities contribute to training a shared model.

Further, in an embodiment, the training can be performed by dividing input data into a training set and a test set. The training set can include a majority of the data (e.g., 80%), and the test set can include the remaining data. Further, the training set itself can be split into two groups: a training group and a validation-on-training group (e.g., split 70/30). The test and validation on training data can be used to predict the trained model's performance under real world conditions.

FIG. 6 illustrates acting on an attrition prediction, according to one embodiment. In an embodiment, a controller 610 generates an attrition intervention event 620. For example, as discussed above in relation to FIG. 3, an attrition service (e.g., the attrition service 162 illustrated in FIGS. 1-2) can use prediction data (e.g., the prediction data 180 illustrated in FIG. 1) with an attrition ML model (e.g., the attrition ML model illustrated in FIGS. 1-2) to predict a probability of attrition. If the probability is sufficiently high (e.g., the probability exceeds a threshold value), the attrition service can trigger attrition intervention event 620 to try to avoid the patient attrition. In an embodiment, the attrition event prophylactically discourages patient attrition from medical treatment, prior to the patient discontinuing medical treatment.

In an embodiment, the controller 610 transmits the attrition intervention event 620, using a communication network 630, to a patient engagement layer 110, a care provider engagement layer 120, or both. The communication network 630 is any suitable communication network, including the Internet, a wide area network, a local area network, or a cellular network, and uses any suitable wired or wireless communication technique (e.g., WiFi or cellular communication).

As discussed above in relation to FIG. 1, the patient engagement layer 110 facilitates engagement with a patient (e.g., through electronic transmissions, telephone calls, and any other suitable techniques) when the patient is predicted to be at risk for attrition from a medical treatment. For example, the patient engagement layer 110 can facilitate transmitting electronic messages (e.g., SMS messages, MMS messages, e-mail messages, electronic notifications, or any other suitable electronic messages) to a patient, and receiving electronic messages from a patient, to facilitate prophylactically reducing a predicted risk of attrition for the patient.

In an embodiment, the care provider engagement layer 120 facilitates engagement with a care provider (e.g., through electronic transmissions to an electronic healthcare system, telephone calls, SMS or MMS messages, and any other suitable techniques) to prophylactically reduce a risk of patient attrition. For example, the care provider engagement layer 120 can engage with the care provider engagement layer 120 to trigger communication from the care provider (e.g., a doctor, nurse, automated system, administrator, or any other suitable entity) to the patient. The care provider can intervene to encourage the patient to continue treatment and avoid attrition.

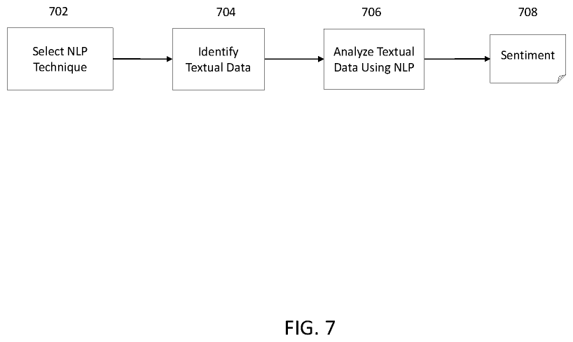

FIG. 7 illustrates parsing data using natural language processing (NLP) for predicting patient attrition using ML, according to one embodiment. In an embodiment, unstructured textual data can be used for prediction of patient attrition. For example, patient notes (e.g., care provider notes or notes written by a patient) can be analyzed using NLP techniques to perform sentiment analysis. The identified sentiment for the notes can be used as part of prediction data (e.g., the prediction data 180 illustrated in FIG. 1) and training data (e.g., the training data 170 illustrated in FIG. 1). In a embodiment, the sentiment analysis is performed as part of a pre-processing step, and the output of the sentiment analysis is used as input for prediction or training data.

In an embodiment, at block 702 a human administer (e.g., a data scientist) or an automated service (e.g., the attrition service 162 illustrated in FIGS. 1-2) selects an NLP technique. For example, the Valence Aware Dictionary for sEntiment Reasoning (VADER) sentiment analysis tool can be used. This is merely an example, and any suitable NLP technique can be used, including any suitable trained ML model (e.g., a logistic regression ML model, a support vector machine (SVM) ML model, a random forest ML model, a naïve Bayes ML model, a deep learning neural network (DNN), a recurrent neural network (RNN), or any other suitable ML model. For example, a suitable ML model can be trained (e.g., using historical notes data) and then used for NLP sentiment analysis.

At block 704, the attrition service, or any other suitable service (e.g., an NLP service), identifies textual data. For example, the attrition service can identify patient notes (e.g., care provider notes or notes written by a patient) including unstructured text.

At block 706, the attrition service analyzes the textual data using NLP. For example, the attrition service can use the NLP technique identified at block 702 to analyze the unstructured text and perform sentiment analysis. In an embodiment, the attrition service generates a sentiment 708, reflecting the sentiment of the textual data identified at block 704. This sentiment can then be used as input to an attrition ML model (e.g., the attrition ML model 164 illustrated in FIG. 2), for prediction and training.

FIG. 8 depicts patient characteristics 800 for use in predicting patient attrition using an ML model, according to one embodiment. In an embodiment, the patient characteristics 800 provide examples for the historical patient data 172, and the patient data 182, described above in relation to FIG. 1. That is, the patient characteristics 800 can be used as features for both training an attrition ML model (e.g., as discussed above in relation to FIG. 5) and predicting a probability of patient attrition using an attrition ML model (e.g., as discussed above in relation to FIGS. 3-4).

A patient 802 includes patient demographics 810. For example, the patient demographics 810 can include age 812 (e.g., at prediction or training time) and gender 814. In an embodiment, the gender 814 can be a binary value (e.g., isMale), a textual value, or any other suitable value.

The patient 802 further includes patient devices 820. For example, the attrition ML model can be used to infer attrition for patients using a therapy involving equipment (e.g., a continuous positive airway pressure (CPAP) machine). The patient devices 820 can identify characteristics of devices used by the patient. A renter 822 field identifies whether the patient owns or rents relevant equipment, A types 824 field identifies the type of equipment used, and a new 826 field identifies whether the patient is new to the relevant therapy. In an embodiment, the types 824 field can be made up of multiple binary values indicating whether the patient uses particular devices. Further, in an embodiment the types 824 field can relate to a brand of equipment preferred by the patient.

The patient 802 further includes patient care 830. For example, the patient care 830 can include data relating to care providers and care facilities associated with the patient. An insurance 832 field indicates insurance information (e.g., whether the patient uses Medicare or Medicaid). A site 834 field indicates a healthcare site for the patient. A site attrition 836 indicates attrition for the site (e.g., an average site-level attrition percentage).

The patient 802 further includes patient diagnoses 840. For example, the patient diagnoses 840 can include a series of diagnosis fields 842A-N (e.g., binary data values) indicating whether a patient has been diagnosed with a particular condition. The diagnoses can relate to a therapy for which the patient is at risk for attrition (e.g., sleep related diagnoses for CPAP therapy), but this is merely an example. The diagnoses can also relate to additional medical diagnoses (e.g., cardiovascular, pulmonary, obesity, infections, fatigue, or any other suitable diagnoses).

The patient 802 further includes patient notes 850. For example, the patient notes 850 can include a count 852 (e.g., a count of notes) and a sentiment 854. The sentiment 854 can describe the sentiment of patient notes (e.g., care provider notes describing a patient or notes written by a patient), as described above in relation to FIG. 7.

In an embodiment, the patient characteristics 800 are merely examples. The patient characteristics 800 can include any suitable data, organized in any suitable fashion and maintained in any suitable manner.

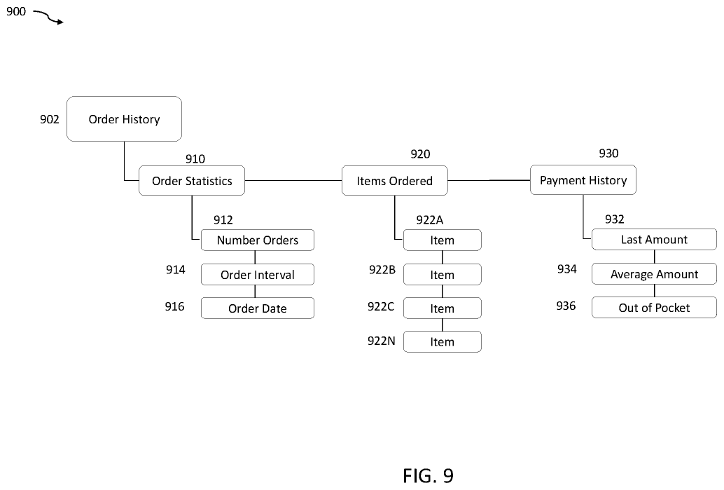

FIG. 9 depicts order history characteristics 900 for use in predicting patient attrition using an ML model, according to one embodiment. In an embodiment, the order history characteristics 900 provide examples for the historical order data 174, and the order data 184, described above in relation to FIG. 1. That is, the order history characteristics 900 can be used as features for both training an attrition ML model (e.g., as discussed above in relation to FIG. 5) and predicting a probability of patient attrition using an attrition ML model (e.g., as discussed above in relation to FIGS. 3-4).

An order history 902 (e.g., for a particular patient) includes order statistics 910. For example, the order statistics 910 can include a number of orders 912 (e.g., a total number of orders for the patient). The order statistics 910 can further include an order interval 914. The order interval 914 can reflect an average time between orders (e.g., a median time), a maximum time between orders, a minimum time between orders, and any other suitable data. The order statistics can further include an order date 916. The order date 916 can include a time since the first order (e.g., a number of months or days), a time since the most recent order (e.g., a number of months or days), and any other suitable information.

The order history 902 further includes items ordered 920. In an embodiment, the items ordered 920 relates to items ordered by the patient in past orders. For example, an item 922A can reflect a quantity (e.g., an average quantity per order) ordered for a particular item (e.g., for CPAP therapy, the items could include nasal cushions, nasal masks, face cushions, face masks, and any other suitable items). The items 922A-N can reflect any suitable number of different items. Further, in an embodiment, the items ordered can include data reflecting the time period when items were purchased (e.g., a percentage within the most recent X months or any other suitable time period).

The order history 902 further includes payment history 930. In an embodiment, the payment history 930 relates to prior payments made by the patient for prior orders. For example, the payment history 930 can include a last amount 932 (e.g., an out of pocket cost for a patient's most recent order) and an average amount 934 (e.g., an average out of pocket cost for the patient, per order). The payment history 930 can further include an out of pocket 936 field. The out of pocket 936 field can reflect how much of the cost of orders is out of pocket for the patient. This can include a percentage of a total cost that was out of pocket for a most recent order, for an average order (e.g., across all orders by the patient), and any other suitable information.

In an embodiment, the order history characteristics 900 are merely examples. The order history characteristics 900 can include any suitable data, organized in any suitable fashion and maintained in any suitable manner.

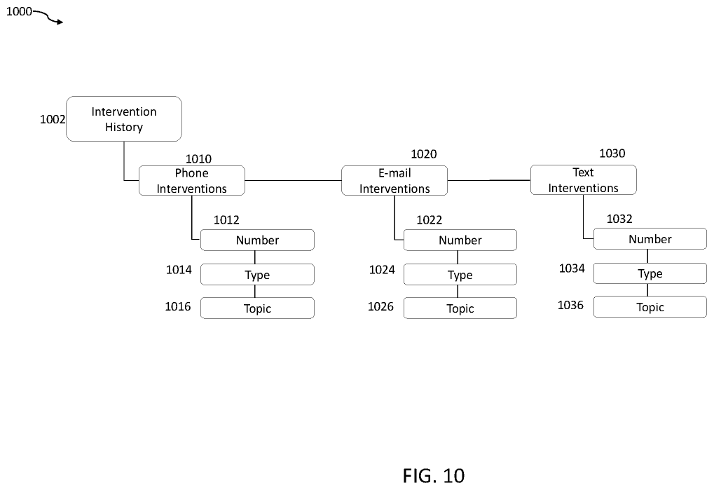

FIG. 10 depicts intervention history characteristics 1000 for use in predicting patient attrition using an ML model, according to one embodiment. In an embodiment, the intervention history characteristics 1000 provide examples for the historical intervention data 176, and the intervention data 186, described above in relation to FIG. 1. That is, the intervention history characteristics 1000 can be used as features for both training an attrition ML model (e.g., as discussed above in relation to FIG. 5) and predicting a probability of patient attrition using an attrition ML model (e.g., as discussed above in relation to FIGS. 3-4).

An intervention history 1002 (e.g., for a particular patient) includes phone interventions 1010. In an embodiment, the phone interventions 1010 relate to interventions made to the patient via telephone, relating to reorder, assistance with a given therapy or treatment, or any other intervention. The phone interventions 1010 includes a number 1012 (e.g., a number of phone interventions) and a type 1014. In an embodiment, the type 1014 indicates a type of telephone call. This can include outbound calls (e.g., to a patient), inbound calls (e.g., from a patient), and any other suitable type. The phone interventions 1010 further include a topic 1016. The topic 1016 can reflect a topic for a telephone intervention (e.g., reorder, therapy assistance, or any other topic). In an embodiment, the type 1014 and topic 1016 are each numerical values reflecting a number of telephone interventions of each available type or associated with each available topic.

The intervention history 1002 further includes e-mail interventions 1020. In an embodiment, the e-mail interventions 1020 relate to interventions made to the patient via e-mail, relating to reorder, assistance with a given therapy or treatment, or any other intervention. The e-mail interventions 1020 includes a number 1022 (e.g., a number of e-mail interventions) and a type 1024. In an embodiment, the type 1024 indicates a type of e-mail interventions 1020. This can include outbound e-mails (e.g., to a patient), inbound e-mails (e.g., from a patient), and any other suitable type. The e-mail interventions 1020 further include a topic 1026. The topic 1026 can reflect a topic for an e-mail intervention (e.g., reorder, therapy assistance, or any other topic). In an embodiment, the type 1024 and topic 1026 are each numerical values reflecting a number of e-mail interventions of each available type or associated with each available topic.

The intervention history 1002 further includes text interventions 1030. In an embodiment, the text interventions 1030 relate to interventions made to the patient via text (e.g., SMS or MMS), relating to reorder, assistance with a given therapy or treatment, or any other intervention. The text interventions 1030 include a number 1032 (e.g., a number of text interventions) and a type 1034. In an embodiment, the type 1034 indicates a type of text interventions 1030. This can include outbound texts (e.g., to a patient), inbound texts (e.g., from a patient), and any other suitable type. The text interventions 1020 further include a topic 1036. The topic 1036 can reflect a topic for a text intervention (e.g., reorder, therapy assistance, or any other topic). In an embodiment, the type 1034 and topic 1036 are each numerical values reflecting a number of text interventions of each available type or associated with each available topic.

In an embodiment, the intervention history characteristics 1000 are merely examples. The intervention history characteristics 1000 can include any suitable data, organized in any suitable fashion and maintained in any suitable manner.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in FIGS., those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A computer-implemented method, comprising:
identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient;
predicting a probability of attrition from a medical treatment for the patient based on providing the prediction data to a machine learning (ML) model,
wherein the ML model is trained to predict the probability using prior patient medical data and prior patient order data, relating to a plurality of prior patients; and
triggering an intervention to prophylactically discourage attrition from the medical treatment for the patient, based on the predicted probability of attrition.

2. The computer-implemented method of claim 1, wherein the prediction data further comprises patient intervention data comprising a plurality of characteristics relating to past interventions with the patient.

3. The computer-implemented method of claim 2, wherein the ML model is trained to predict the probability further using prior patient intervention data.

4. The computer-implemented method of claim 2, wherein the patient medical data comprises two or more of: (i) demographic data for the patient, (ii) medical equipment data for the patient, (iii) care provider data for the patient, and (iv) prior diagnosis information for the patient.

5. The computer-implemented method of claim 4, wherein the patient medical data further comprises sentiment analysis generated using natural language processing (NLP) for one or more medical notes relating to the patient.

6. The computer-implemented method of claim 2, wherein the patient order data comprises two or more of: (i) statistical information for the order history for medical items relating to the patient, (ii) information describing items previously ordered by the patient as part of the order history, and (iii) payment history information relating to the order history for medical items relating to the patient.

7. The computer-implemented method of claim 1, wherein triggering the intervention comprises triggering an automated communication to at least one of: (i) the patient, (ii) a care provider associated with the patient, or (iii) a care facility associated with the patient.

8. The computer-implemented method of claim 7, wherein the automated communication comprises at least one of: (i) an automated telephone call, (ii) a short message service (SMS) message, (iii) a multimedia messaging service message (MMS), or (iv) an e-mail message.

9. The computer-implemented method of claim 1, wherein the automated communication comprises an electronic communication to the patient.

10. The computer-implemented method of claim 1, wherein triggering an intervention to prophylactically discourage attrition from the medical treatment for the patient, based on the predicted probability of attrition comprises:
determining that the predicted probability of attrition exceeds a threshold value, and in response triggering the intervention.

11. An apparatus comprising:
a memory; and
a hardware processor communicatively coupled to the memory, the hardware processor configured to perform operations comprising:
identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient;
predicting a probability of attrition from a medical treatment for the patient based on providing the prediction data to a machine learning (ML) model,
wherein the ML model is trained to predict the probability using prior patient medical data and prior patient order data, relating to a plurality of prior patients; and
triggering an intervention to prophylactically discourage attrition from the medical treatment for the patient, based on the predicted probability of attrition.

12. The apparatus of claim 11, wherein the prediction data further comprises patient intervention data comprising a plurality of characteristics relating to past interventions with the patient.

13. The apparatus of claim 12,
wherein the patient medical data comprises two or more of: (i) demographic data for the patient, (ii) medical equipment data for the patient, (iii) care provider data for the patient, and (iv) prior diagnosis information for the patient, and
wherein the patient order data comprises two or more of: (i) statistical information for the order history for medical items relating to the patient, (ii) information describing items previously ordered by the patient as part of the order history, and (iii) payment history information relating to the order history for medical items relating to the patient.

14. The apparatus of claim 13, wherein the patient medical data further comprises sentiment analysis generated using natural language processing (NLP) for one or more medical notes relating to the patient.

15. The apparatus of claim 11, wherein triggering an intervention to prophylactically discourage attrition from the medical treatment for the patient, based on the predicted probability of attrition comprises:
determining that the predicted probability of attrition exceeds a threshold value, and in response triggering the intervention.

16. A non-transitory computer-readable medium comprising instructions that, when executed by a processor, cause the processor to perform operations comprising:
identifying a plurality of prediction data, the prediction data comprising both: (i) patient medical data comprising a plurality of characteristics relating to a medical history for the patient, and (ii) patient order data comprising a plurality of characteristics relating to an order history for medical items relating to the patient;
predicting a probability of attrition from a medical treatment for the patient based on providing the prediction data to a machine learning (ML) model,
wherein the ML model is trained to predict the probability using prior patient medical data and prior patient order data, relating to a plurality of prior patients; and
triggering an intervention to prophylactically discourage attrition from the medical treatment for the patient, based on the predicted probability of attrition.

17. The non-transitory computer-readable medium of claim 16, wherein the prediction data further comprises patient intervention data comprising a plurality of characteristics relating to past interventions with the patient.

18. The non-transitory computer-readable medium of claim 17,
wherein the patient medical data comprises two or more of: (i) demographic data for the patient, (ii) medical equipment data for the patient, (iii) care provider data for the patient, and (iv) prior diagnosis information for the patient, and
wherein the patient order data comprises two or more of: (i) statistical information for the order history for medical items relating to the patient, (ii) information describing items previously ordered by the patient as part of the order history, and (iii) payment history information relating to the order history for medical items relating to the patient.

19. The non-transitory computer-readable medium of claim 18, wherein the patient medical data further comprises sentiment analysis generated using natural language processing (NLP) for one or more medical notes relating to the patient.

20. The non-transitory computer-readable medium of claim 16, wherein triggering an intervention to prophylactically discourage attrition from the medical treatment for the patient, based on the predicted probability of attrition comprises:
determining that the predicted probability of attrition exceeds a threshold value, and in response triggering the intervention.
